(19) United States
(12) Patent Application Publication
(10) Pub. No.: US 2025/0303157 A1
(43) Pub. Date: Oct. 2, 2025

(54) ELECTRIC STIMULATION MASSAGER AND ELECTRIC STIMULATION MASSAGING DEVICE

(71) Applicant: SKG Health Technologies Co., Ltd., Shenzhen (CN)

(72) Inventors: Junchi WU, Shenzhen (CN); Yunfei ZHU, Shenzhen (CN)

(73) Assignee: SKG Health Technologies Co., Ltd., Shenzhen (CN)

(21) Appl. No.: 19/016,625

(22) Filed: Jan. 10, 2025

(30) Foreign Application Priority Data
Apr. 1, 2024 (CN) 202410390276.9

(51) Int. Cl. A61N 1/36; H03K 3/017

(52) U.S. Cl. CPC A61N 1/36003; A61N 1/36021; A61N 1/36034; H03K 3/017

(57) ABSTRACT

An electric stimulation massager and an electric stimulation massaging device are provided. The electric stimulation massager includes a pulse output portion, a base body, multiple electrode groups, and a controller. The pulse output portion is configured to output an electrical pulse, where the pulse output portion includes multiple electrical connection terminals. Each of the multiple electrode groups includes at least one electrode electrically connected to a same electrical connection terminal, and the multiple electrode groups are spaced apart from one another and arranged in a loop. The controller is configured to form conduction of at least two electrical connection terminals of the pulse output portion, to enable the at least two electrical connection terminals to be in electrical conduction with a human body through corresponding electrode groups to perform electric stimulation massage on the human body.

### CROSS-REFERENCE TO RELATED APPLICATIONS

This application claims priority under 35 U.S.C. § 119(a) to and the benefit of Chinese Patent Application No. 202410390276.9, filed Apr. 1, 2024, the entire disclosure of which is incorporated herein by reference.

### TECHNICAL FIELD

The application relates to the field of electric stimulation massager, and in particular, to an electric stimulation massager and an electric stimulation massaging device.

### BACKGROUND

Electrical impulse massagers have advantages such as being compact, portable, and having adjustable massage intensity, making them better suited to various lifestyle scenarios and user needs. In the related art, the electrical impulse massagers achieve muscle massage and fatigue relief by means of electrical stimulation. However, the massage effect produced is singular in nature and can easily cause user fatigue.

### SUMMARY

In a first aspect, an electric stimulation massager is provided in the embodiments of the disclosure. The electric stimulation massager includes a pulse output portion, a base body, multiple electrode groups disposed on one side of the base body, and a controller. The pulse output portion is configured to output an electrical pulse, where the pulse output portion includes multiple electrical connection terminals. Each of the multiple electrode groups includes at least one electrode electrically connected to a same electrical connection terminal, and the multiple electrode groups are spaced apart from one another and arranged in a loop. The controller is configured to form conduction of at least two electrical connection terminals of the pulse output portion, to enable the at least two electrical connection terminals to be in electrical conduction with a human body through corresponding electrode groups to perform electric stimulation massage on the human body. The controller is further configured to control the pulse output portion to switch conduction of the multiple electrical connection terminals in a preset sequence, to sequentially conduct the multiple electrode groups in a clockwise or counterclockwise direction to perform a circular massage on the human body.

In a second aspect, an electric stimulation massaging device is provided in the embodiments of the disclosure. The electric stimulation massaging device includes a strap and the electric stimulation massager according to any one of the above embodiments, where the electric stimulation massager is connected to the strap.

Additional aspects and advantages of the disclosure will be partially set forth in the following description, will become apparent from the following description, or will be learned through practice of the disclosure.

### BRIEF DESCRIPTION OF THE DRAWINGS

The above and/or additional aspects and advantages of the disclosure will become apparent and readily understood from the description of embodiments in conjunction with the accompanying drawings.

FIG. 1 is a schematic diagram of an electrical stimulation massager according to some embodiments of the disclosure.

FIG. 2 is a schematic diagram of a pulse output portion and electrode groups according to some embodiments of the disclosure.

FIG. 3 is a schematic diagram of an electrical stimulation massager according to some embodiments of the disclosure.

FIG. 4 is a schematic diagram illustrating switching conduction of multiple electrode groups according to some embodiments of the disclosure.

FIG. 5 is a schematic diagram illustrating switching conduction of multiple electrode groups according to some embodiments of the disclosure.

### DETAILED DESCRIPTION

Embodiments of the disclosure will be described in detail below, with these embodiments illustrated in the accompanying drawings, where identical or similar reference numerals throughout denote identical or similar elements, or elements having identical or similar functions. The embodiments described below with reference to the accompanying drawings are exemplary and are provided solely to explain the disclosure, and should not be construed as limiting the disclosure.

Electrical impulse massagers have advantages such as being compact, portable, and having adjustable massage intensity, making them better suited to various lifestyle scenarios and user needs. In the related art, the electrical impulse massagers achieve muscle massage and fatigue relief by means of electrical stimulation. However, the massage effect produced is singular in nature and can easily cause user fatigue.

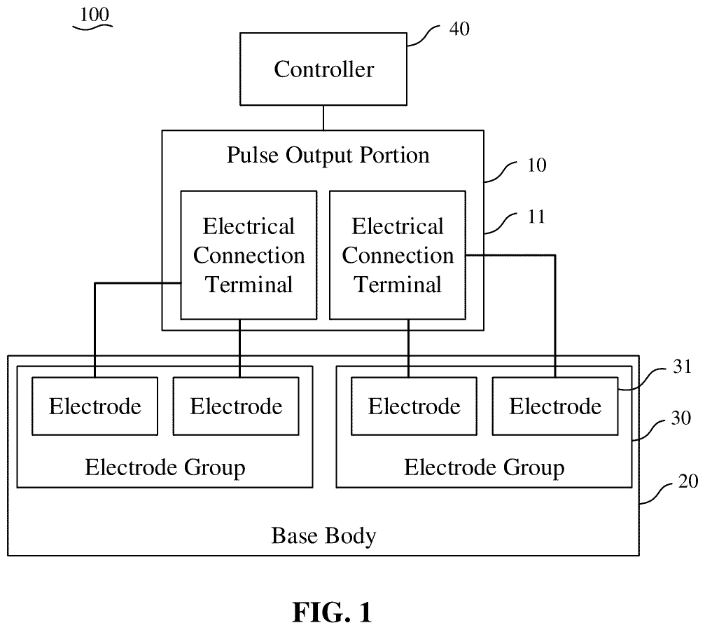

Referring to FIGS. 1 to 3, an electric stimulation massager 100 is provided in the embodiments of the disclosure. The electric stimulation massager 100 includes a pulse output portion 10, a base body 20, multiple electrode groups 30 disposed on one side of the base body 20, and a controller 40. The pulse output portion 10 is configured to output an electrical pulse, where the pulse output portion 10 includes multiple electrical connection terminals 11. Each of the multiple electrode groups 30 includes at least one electrode 31 electrically connected to a same electrical connection terminal 11 among the multiple electrical connection terminals, and the multiple electrode groups 30 are spaced apart from one another and arranged in a loop. The controller 40 is configured to form conduction of at least two electrical connection terminals 11 among the multiple electrical connection terminals of the pulse output portion 10, to enable the at least two electrical connection terminals 11 to be in electrical conduction with a human body through corresponding electrode groups 30 to perform electric stimulation massage on the human body. The controller 40 is further configured to control the pulse output portion 10 to switch conduction of the multiple electrical connection terminals 11 in a preset sequence, to sequentially conduct the multiple electrode groups 30 in a clockwise or counterclockwise direction to perform a circular massage (also known as rotational massage) on the human body.

Specifically, the electrical connection terminal 11 of the pulse output portion 10 is electrically connected to at least one electrode 31. Electrodes electrically connected to the same electrical connection terminal 11 form the electrode group 30. The multiple electrode groups 30 are spaced apart from one another to avoid mutual interference during conduction. The multiple electrode groups 30 may be arranged in a closed loop, such as a circle, triangle, quadrilateral, ellipse, polygon, or other shapes. The controller 40 is configured to form conduction of the at least two electrical connection terminals 11 of the pulse output portion 10. The electrical connection terminal 11 may be contacted to the human body through the electrode group 30. The human body may serve as a conductor. The electrical connection terminal 11 conducted is in electrical conduction with the human body through the electrode group 30. When the electrical connection terminal 11 is conducted, at least one electrical connection terminal 11 delivers current to the human body via the electrode group 30 to stimulate human muscles, thereby producing a massage sensation. At least one electrical connection terminal 11 is at a low level to form an electrical conduction with the electrical connection terminal 11 conducted via the electrode group 30 and the human body. The controller 40 is further configured to control the pulse output portion 10 to switch the conduction of the electrical connection terminals 11 in the preset sequence. That is, the controller 40 may stop conduction of at least one electrical connection terminal 11 that is conducted at an earlier time and conduct at least one another electrical connection terminal 11. The at least one another electrical connection terminal 11 that is conducted is adjacent to the at least one electrical connection terminal 11 that is conducted at an earlier time in a clockwise or counterclockwise direction, thereby enabling the multiple electrode groups 30 to be sequentially conducted in the clockwise or counterclockwise direction to produce a circular massage effect on the human body. In one embodiment, referring to FIG. 4, the controller 40 sequentially switches electrical conduction between electrical connection terminals 11 connected to electrode group A and electrical connection terminals 11 connected to electrode group B, electrical conduction between the electrical connection terminals 11 connected to electrode group B and electrical connection terminals 11 connected to electrode group C, electrical conduction between electrical connection terminals 11 connected to electrode group C and electrical connection terminals 11 connected to electrode group D, and electrical conduction between electrical connection terminals 11 connected to electrode group D and the electrical connection terminals 11 connected to electrode group A, thereby sequentially conduct the multiple electrode groups 30 in the clockwise direction to produce a clockwise circular massage effect on the human body. Thus, by controlling the pulse output portion 10 to sequentially switch conduction of the electrical connection terminals 11 in a preset order and direction, a circular massage technique can be simulated, thereby creating a unique massage experience.

In some embodiments, at least two electrode groups 30 among the multiple electrode groups 30 are simultaneously conducted, at least two electrode groups 30 conducted at an earlier time and at least two electrode groups 30 conducted later in sequence share at least one same electrode group 30.

Specifically, the pulse output portion 10 switches conduction of the electrical connection terminals 11, thereby enabling at least one different electrical connection terminal 11 to be in contact with and in electrical conduction with the human body through the electrode group 30. At least two electrical connection terminals 11 among the multiple electrical connection terminals 11 are simultaneously conducted, at least two electrical connection terminals 11 conducted at an earlier time and at least two electrical connection terminals 11 conducted later in sequence share at least one same electrical connection terminal 11. This ensures that at least two electrode groups 30 among the multiple electrode groups 30 are simultaneously conducted, at least two electrode groups 30 conducted at an earlier time and at least two electrode groups 30 conducted later in sequence share at least one same electrode group 30, thereby providing a more continuous circular massage sensation for a user and better simulating the circular massage sensation during formation of a circular massage. In one embodiment, referring to FIG. 4, the controller 40 first controls the electrical connection terminal 11 connected to electrode group A and the electrical connection terminal 11 connected to electrode group B to be in electrical conduction with one another, and then switches to control the electrical connection terminals 11 connected to electrode group B and the electrical connection terminals 11 connected to electrode group C to be in electrical conduction with one another. In both of these two successive electrical conduction, the electrical connection terminal 11 connected to electrode group B remains conductive.

At least two electrode groups 30 among the multiple electrode groups are simultaneously conducted, where at least two electrode groups 30 conducted at an earlier time and at least two electrode groups conducted later in sequence share at least one same electrode group 30, thereby allowing for a more continuous user sensation to align closely with the experience of circular massage.

In some embodiments, the controller 40 is further configured to control the pulse output portion 10 to output the electrical pulse through at least one electrical connection terminal 11 and to control at least one electrical connection terminal 11 to be grounded, to enable at least two electrical connection terminals 11 to be in electrical conduction with one another.

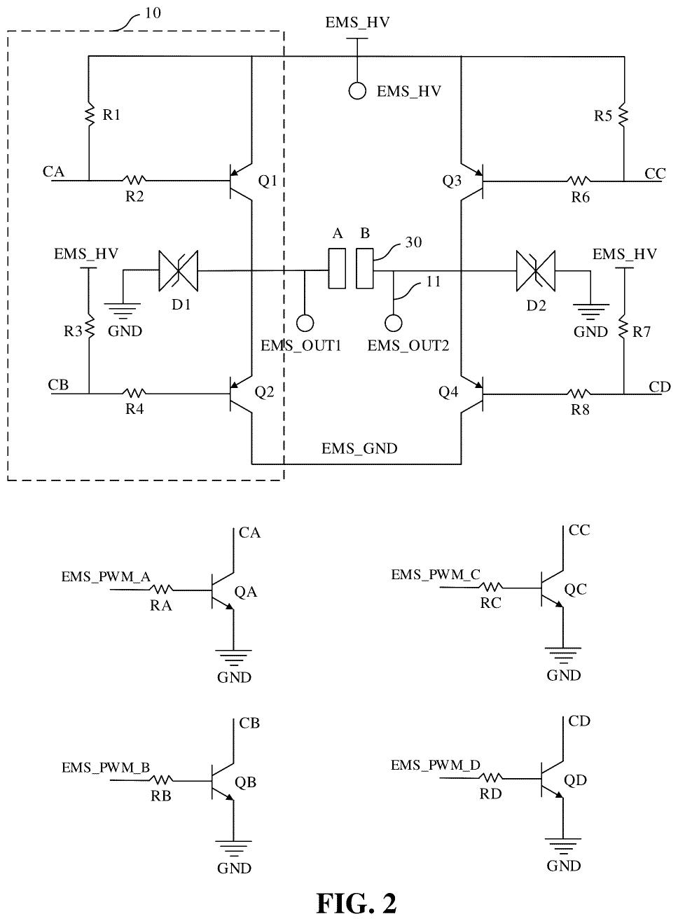

Specifically, the controller 40 controls the pulse output portion 10 to output the electrical pulse through the at least one electrical connection terminal 11, allowing the electrical connection terminal 11 to deliver current to the human body through the electrode group 30 connected to the electrical connection terminal 11, where the electrode group 30 connected to the electrical connection terminal 11 serves as an output electrode group 32. The controller 40 also controls the at least one electrical connection terminal 11 to be grounded, enabling current to flow through the human body into the electrical connection terminal 11 grounded, where the electrode group 30 connected to the electrical connection terminal 11 grounded serves as an input electrode group 33. When the electrical connection terminal 11 through which the electrical pulse is outputted and the electrical connection terminal 11 that is grounded are conducted, a voltage difference is established between the two electrical connection terminals 11. Since the human body serves as a conductor, electrical conduction is formed between the two electrical connection terminals 11 through the output electrode group 32, the input electrode group 33, and the human body. Referring to FIG. 2, in one embodiment, the pulse output portion forms an H-bridge corresponding to two electrical connection terminals. For example, if there are four electrical connection terminals, two H-bridges are formed to correspondingly connect the electrical connection terminals for outputting of electrical pulses. The controller 40 controls conduction of electrical connection terminal A and electrical connection terminal B by controlling conduction of switching transistors in the H-bridge connected to electrical connection terminal A and electrical connection terminal B. The controller 40 controls conduction of transistor QA to turn on transistor Q1 and controls conduction of transistor QB to turn off transistor Q2, thereby enabling the electrical connection terminal 11 connected to electrode group A to be connected to voltage input EMS_HV to deliver an electrical pulse. Simultaneously, the controller 40 controls conduction of transistor QC to turn off transistor Q3 and controls conduction of transistor QD to turn on transistor Q4, thereby grounding the electrical connection terminal 11 connected to electrode group B. This configuration enables conduction of the electrical connection terminal 11 connected to electrode group A and conduction of the electrical connection terminal 11 connected to electrode group B. Referring to FIG. 4, an electrical pulse may be outputted through the electrical connection terminal 11 connected to the electrode group 30 with grid shading, and the electrical connection terminal 11 connected to the electrode group 30 with horizontal-line shading is grounded. The controller 40 sequentially switches the electrical connection terminal 11 through which an electrical pulse is outputted and the electrical connection terminal 11 that is grounded, the multiple electrode groups 30 can be sequentially conducted in the clockwise direction, thereby performing circular massage to the human body. In other words, the controller 40 sequentially switches the electrical connection terminal 11 between outputting an electrical pulse and being grounded, enabling the multiple electrode groups 30 to be sequentially conducted in a clockwise direction, thereby performing a rotational massage on the human body.

In this way, by controlling at least one electrical connection terminal 11 to output an electrical pulse and at least one electrical connection terminal 11 to be grounded, the electrical connection terminals 11 can be in electrical conduction with one another, an electrical conduction between the electrical connection terminals 11 and the human body through the electrode groups 30 connected with the electrical connection terminals 11 can be formed, thereby achieving the effect of electrical stimulation massage.

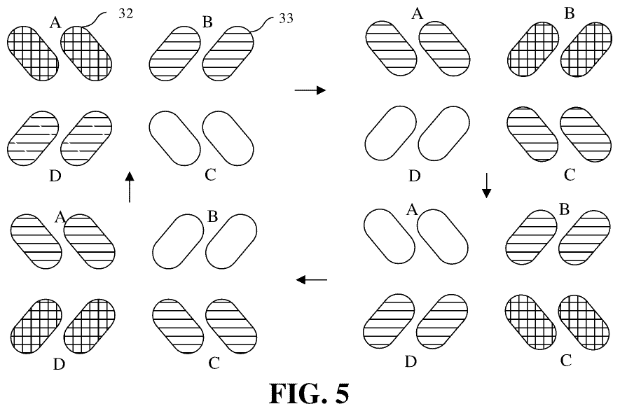

Referring to FIG. 5, in some embodiments, among the electrical connection terminals 11 that are in electrical conduction with one another, the number (quantity) of electrodes connected to the electrical connection terminal 11 through which the electrical pulse is outputted is less than the number (quantity) of electrodes 31 connected to the electrical connection terminal 11 that is grounded.

Specifically, the human body experiences a stronger physical sensation at the position of the electrode group 30 connected to the electrical connection terminal 11 through which an electrical pulse is outputted. Therefore, by controlling the number (quantity) of electrodes connected to the electrical connection terminal 11 through which an electrical pulse is outputted to be smaller than the number (quantity) of electrodes connected to the electrical connection terminals 11 that are grounded, a stronger current to be outputted through the electrical connection terminal 11 through which an electrical pulse can be outputted, thereby creating a stronger massage effect on the human body. In one embodiment, referring to FIG. 5, the controller 40 controls an electrical pulse to be outputted through the electrical connection terminal 11 connected to electrode group A with grid shading, and controls the electrical connection terminals 11 connected to electrode groups B and D with horizontal-line shading to be grounded, ensuring that the electrical pulse is outputted through one electrical connection terminal 11 while two electrical connection terminals 11 are grounded.

In this way, by controlling the number (quantity) of electrodes 31 connected to the electrical connection terminal 11 through which the electrical pulse is outputted to be less than the number (quantity) of electrodes 31 connected to the electrical connection terminal 11 that is grounded, allowing the user to experience a stronger circular massage effect.

In some embodiments, each of the multiple electrode groups 30 includes the same number of electrodes 31, the controller 40 is configured to control the pulse output portion 10 to output an electrical pulse through one electrical connection terminal 11 among the multiple electrical connection terminals 11 and to control two electrical connection terminals 11 among the multiple electrical connection terminals to be grounded, and each of the at least one electrode 31 of each of the multiple electrode groups 30 has an area ranging from 1500 mm² to 2500 mm².

Specifically, the controller 40 is configured to control the pulse output portion 10 to output the electrical pulse through one electrical connection terminal 11 and to control two electrical connection terminals 11 to be grounded, resulting in one output electrode group 32 and two input electrode groups 33, enabling a stronger massage effect on the human body. The larger the area of the electrode 31, the greater the current the electrode 31 can carry. However, if the area of the electrode 31 is too large, unintended muscles may be stimulated. Therefore, the area of the electrode 31 is controlled within a range of 1500 mm² to 2500 mm² to support an operating condition in which the electrical pulse is outputted through one electrical connection terminal 11 while two electrical connection terminals 11 are grounded.

In this way, controlling the area of the electrode 31 connected to the electrical connection terminal 11 through which the electrical pulse is outputted within a range of 1500 mm² to 2500 mm² enables the operating condition in which the electrical pulse is outputted through one electrical connection terminal 11 while two electrical connection terminals 11 are grounded.

In some embodiments, among the electrical connection terminals 11 that are in electrical in conduction with one another, the number (quantity) of electrodes 31 connected to the electrical connection terminal 11 through which the electrical pulse is outputted is equal to the number (quantity) of electrodes 31 connected to the electrical connection terminal 11 that is grounded.

Specifically, among the electrical connection terminals 11 that are in electrical conduction with one another, each electrical connection terminal 11 is connected to the same number of electrodes 31. The number of electrical connection terminals 11 through which the electrical pulse is outputted is equal to the number of electrical connection terminals 11 that are grounded, thereby ensuring that the number of electrodes 31 in the output electrode group 32 is the same as the number of electrodes 31 in the input electrode group 33.

In this way, by controlling the number (quantity) of electrodes 31 connected to the electrical connection terminal 11 through which the electrical pulse is outputted and the number (quantity) of electrodes 31 connected to the electrical connection terminals 11 that are grounded, a more uniform sensation can be provided for the user, reducing strong stimulation.

In some embodiments, an operation process of the electric stimulation massager 100 includes an initial phase and an intermediate phase. An electrode group 30 connected to the electrical connection terminal 11 through which the electrical pulse is outputted serves as an output electrode group 32, and an electrode group 30 connected to the electrical connection terminal 11 that is grounded serves as an input electrode group 33. In the initial phase, a distance between any one output electrode group 32 among the at least one output electrode group 30 and any one input electrode group 33 among the at least one input electrode group 30 that are simultaneously conducted is less than a preset distance. In the intermediate phase, a distance between the at least one output electrode group 32 and the at least one input electrode group 33 that are simultaneously conducted is greater than the preset distance.

Specifically, the greater the distance between the output electrode group 32 and the input electrode group 33, the deeper the stimulation applied to muscles of the human body, resulting in a stronger massage sensation. During the initial phase of the operation of the electric stimulation massager 100, the human body requires a period to adapt to the massage. If strong stimulation is applied during the initial phase, it may cause discomfort. Therefore, during the initial phase, the distance any one output electrode group 32 and any one input electrode group 33 that are simultaneously conducted is set to be less than the preset distance. During the intermediate phase, the human body has adapted to the massage stimulation; hence, to achieve a better massage effect, the distance between any one output electrode group 32 and any one input electrode group 33 that are simultaneously conducted can be greater than the preset distance, thereby providing a stronger massage sensation and generating a stronger massage force.

In this way, because the stimulation experienced by the user is relatively mild when the output electrode group 32 is relatively close to the input electrode group 33, electrode groups 30 that are positioned relatively close to each other are selected for conduction during the initial phase of the massage, serving as a warm-up. During the intermediate phase of the massage, electrode groups 30 that are positioned relatively far from each other are selected for conduction to provide the user with stronger massage stimulation.

In some embodiments, the controller 40 is configured to control the multiple electrical connection terminals 11 to be conducted sequentially at an interval ranging from 50 ms to 500 ms.

Specifically, if a duration of a single conduction of the electrical connection terminal 11 is too long, it results in a prolonged time of a prolonged massage on a single point of on the human body. The human body will experience continuous massage at a position where the electrode group 30 connected to the currently conducted electrical connection terminal 11 is in contact with the human body. This results in a continuous massage at a single point, and when conduction is switched to another electrical connection terminal 11, the human body can only perceive a shift in the position of continuous single-point massage, failing to create a circular massage sensation. If the duration of a single conduction of the electrical connection terminal 11 is too short, the muscles of the human body will not experience a massage sensation under the electrical stimulation, resulting in a disorganized physical sensation. Therefore, when the multiple electrical connection terminals 11 are conducted sequentially at an interval ranging from 50 ms to 500 ms, a circular massage effect can be effectively simulated.

In this way, when the interval between each two adjacent switching falls within the range of 50 ms to 500 ms, the electric stimulation massager 100 can effectively simulate the circular massage sensation.

In some embodiments, the controller 40 is configured to control the multiple electrical connection terminals 11 to be conducted sequentially at an interval ranging from 200 ms to 400 ms.

Specifically, the controller 40 is configured to control the multiple electrical connection terminals 11 to be conducted sequentially at the interval ranging from 200 ms to 400 ms, allowing for a moderate interval for sequentially conducting the electrical connection terminals 11, thereby providing a better circular massage sensation for the user.

In this way, when the interval between each two adjacent switching falls within the range of 200 ms to 400 ms, the electric stimulation massager 100 can better simulate a circular massage effect. This avoids excessively short conduction time, which may cause a disorganized physical sensation for the user, and excessively long conduction time, which may result in the electrode group 30 switching too slowly and the absence of a circular sensation.

In some embodiments, an envelope waveform of the electrical pulse includes a sine waveform, a triangular waveform, or a trapezoidal waveform.

Specifically, in the case of the electrical pulse with an envelope in the form of a sine wave, triangular wave, or trapezoidal wave, within a single envelope waveform, the time for the amplitude to increase from the median value to the peak value is relatively long, and the time for the amplitude to decrease from the peak value back to the median value is also relatively long. This ensures that the electrical pulse has a gradual change during both the initial and terminal phases within a single envelope waveform. Consequently, when the electrical pulse is applied to the human body through the electrode group 30, the intensity of the current output to the human body also has a gradual change. This provides a gentler physical sensation during switching of conduction of the electrical connection terminals 11, avoiding sudden stimulation and thereby better simulating the circular massage sensation.

In this way, an electrical pulse forming envelope waveforms such as a sine wave, a triangular wave, or a trapezoidal wave has a relatively long time for the amplitude to increase from the median value to the peak value, allowing the electrical pulse to have a gradual change. Consequently, when the electrical pulse is applied through the electrode group 30, the intensity of the current outputted through the electrode group 30 may also have a gradual change, enabling the switching of conduction of the electrodes 31 to be relatively smooth and comfortable.

In some embodiments, a frequency of the electrical pulse ranges from 1 kHz to 6 kHz, and a pulse width of the electrical pulse ranges from 40 μs to 250 μs.

Specifically, the frequency of the electrical pulse is controlled to range from 1 kHz to 6 kHz, and the pulse width of the electrical pulse is controlled to range from 40 μs to 250 μs. This ensures that when the electrical pulse is applied to the electrode group 30 and current is outputted through the electrode group 30 to the human body to stimulate muscles, the muscles may experience a sensation similar to the sensation of kneading massage, thereby simulating massage techniques.

In this way, by limiting the frequency of the electrical pulse to a range of 1 kHz to 6 kHz and the pulse width of the electrical pulse to a range of 40 μs to 250 μs, the physical sensation experienced by the muscles when the electrical pulse is applied to the human body becomes more similar to the sensation of a kneading massage. This simulates the kneading technique, providing the user with an experience that more resembles actual kneading massage.

In some embodiments, the electric stimulation massager 100 is connected to a strap 200, the electrode 31 is strip-shaped, and an included angle between a longitudinal direction of the electrode 31 and a longitudinal direction of the strap 200 ranges from 30 degrees to 60 degrees.

Specifically, if the included angle between the longitudinal direction of the electrode 31 and the longitudinal direction of the strap 200 is too large or too small, the adjacent electrodes 31 may align to form a straight line. When the electrode group 30 conducts current, the current may preferentially flow into ends of electrodes 31 that are closer to one another, resulting in uneven current distribution. This causes electrodes 31 with a relatively small spacing to provide stronger stimulation to the user. Therefore, setting the included angle between the longitudinal direction of the electrode 31 and the longitudinal direction of the strap 200 within a range of 30 degrees to 60 degrees can prevent uneven current distribution, providing the user with a more uniform massage sensation.

In this way, if the included angle between the longitudinal direction of the electrode 31 and the longitudinal direction of the strap 200 is too large or too small, the electrodes 31 may align into a straight line. This causes the current to flow preferentially through the ends of the electrodes 31 that are closer to one another during conduction, leading to uneven distribution. By setting the angle of inclination of the electrodes 31 relative to the longitudinal direction of the strap 200 within the range of 30 degrees to 60 degrees, the current distribution becomes more uniform, resulting in an improved physical sensation for the user.

In some embodiments, the electrode is strip-shaped, and in each two adjacent electrode groups 30 among the multiple electrode groups 30, an included angle between a longitudinal direction of an electrode 31 in one electrode group 30 and a longitudinal direction of an electrode 31 in another electrode group 30 ranges from 60 degrees to 90 degrees.

Specifically, setting the included angle between the longitudinal direction of an electrode 31 in one electrode group 30 and the longitudinal direction of an electrode 31 in another electrode group 30 adjacent to the one electrode group 30 ranges from 60 degrees to 90 degrees can prevent the electrodes 31 in each two adjacent electrode groups 30 among the multiple electrode groups 30 from aligning in a straight line in the longitudinal direction. This helps avoid uneven current distribution, thereby providing a better massage sensation for the user.

In this way, by controlling the included angle between the longitudinal direction of an electrode 31 in one electrode group 30 and the longitudinal direction of an electrode 31 in another electrode group 30 adjacent to the one electrode group 30 ranges from 60 degrees to 90 degrees, the current conduction in the electrodes 31 becomes more concentrated, improving physical sensation of the user.

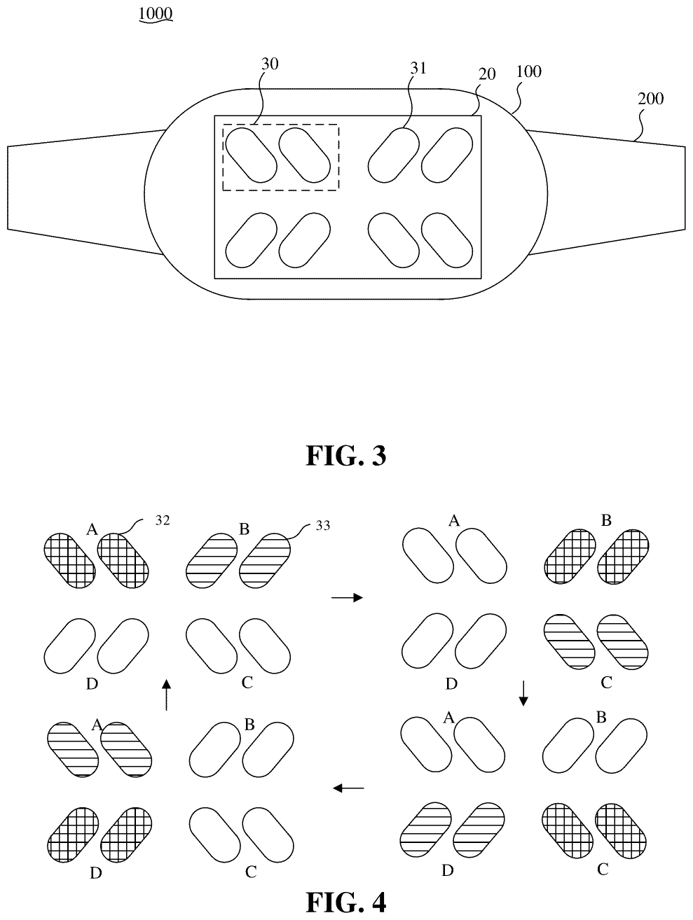

Referring to FIG. 3, an electric stimulation massaging device 1000 is provided in the embodiments of the disclosure. The electric stimulation massaging device 1000 includes a strap 200 and the electric stimulation massager 100 according to any one of the above embodiments, where the electric stimulation massager 100 is connected to the strap 200.

In this way, by controlling the pulse output portion 10 to sequentially switch conduction of the electrical connection terminals 11 in a preset order and a preset direction, a circular massage technique can be simulated, thereby creating a unique massage experience.

In some embodiments, the electric stimulation massaging device 1000 is a waist

massaging device. The multiple electrode groups 30 are arranged in two loops, spaced apart from one another, and arranged to correspond to an erector spinae and a quadratus lumborum of the human body, to perform a circular massage on the erector spinae and the quadratus lumborum on both sides of the human body.

Specifically, the lumbar massager includes multiple electrode groups 30, which are arranged in two loops, spaced apart from one another, and arranged to correspond to the erector spinae and quadratus lumborum muscles of the human body. When the controller 40 controls the electrical connection terminal 11 to be conducted, the electrical connection terminal 11 conducted comes into electrical conduction with the human body through the electrode group 30 connected to the electrical connection terminal 11. The electrical pulse outputted through the electrical connection terminal 11 is applied through the electrode group 30, current is delivered by the electrode group 30 to the human body to electrically stimulate the erector spinae and the quadratus lumborum on both sides of the human body, thereby simulating a massage sensation. The controller 40 switches conduction of the electrical connection terminals 11 so that the electrode groups 30 are sequentially conducted in the clockwise or counterclockwise manner, creating a circular massage sensation for the erector spinae and the quadratus lumborum on both sides of the body.

In this way, the electrode groups 30 are spaced apart from one another and correspond to the the erector spinae and the quadratus lumborum, allowing the electrical connection terminals 11 to be in electrical conduction with the erector spinae and the quadratus lumborum through the electrode groups 30, thereby stimulating and providing a circular massage for the erector spinae and the quadratus lumborum of the human body.

In the description of the specification, reference terms such as “some embodiments”, “some embodiments”, “exemplary embodiment”, “example,” “specific example”, or “certain examples” referred to herein mean that a particular feature, structure, material, or characteristic described in connection with the embodiment or example is included in at least one embodiment or example of the disclosure. In the specification, the indicative expressions of these terms do not necessarily refer to the same embodiment or example. Furthermore, without conflicting with one another, a person skilled in the art may combine and integrate different embodiments or examples and features of different embodiments or examples described in the specification.

Furthermore, the term “connection” should be broadly interpreted to include, for example, fixed connections, detachable connections, or integral connections. It may include direct connections, indirect connections via intermediate media, and also communication within two components. A person of ordinary skill in the art can understand the specific meaning of the above terms in the disclosure according to the specific circumstances.

Additionally, the terms “first” and “second” are used solely for descriptive purposes and should not be understood to indicate or imply relative importance, nor to implicitly specify the number of technical features referred herein. Accordingly, features designated as “first” or “second” may explicitly or implicitly include at least one of the features. In the context of the disclosure, “multiple” refers to at least two, such as two, three, etc., unless otherwise specifically defined.

Any process or method described in the flowchart or otherwise herein may be understood as representing a module, segment, or portion of executable instruction code that includes one or more instructions for implementing specific logical functions, or steps of a process. The scope of preferred embodiments of the disclosure includes additional implementations in which functions may be performed in an order different from that shown or discussed, including executing functions in a substantially simultaneous manner or in reverse order, based on the functionality involved, as would be understood by those skilled in the art to which the embodiments of the disclosure pertain.

Although the embodiments of the disclosure have been illustrated and described above, it is understood that these embodiments are exemplary and should not be construed as limiting the disclosure. Those skilled in the art may make variations, modifications, replacements, and variations to the embodiments described above within the scope of the disclosure.

What is claimed is:

1. An electric stimulation massager, comprising:
a pulse output portion configured to output an electrical pulse, wherein the pulse output portion comprises a plurality of electrical connection terminals;
a base body;
a plurality of electrode groups disposed on one side of the base body, wherein each of the plurality of electrode groups comprises at least one electrode electrically connected to a same electrical connection terminal among the plurality of electrical connection terminals, and the plurality of electrode groups are spaced apart from one another and arranged in a loop; and
a controller configured to form conduction of at least two electrical connection terminals among the plurality of electrical connection terminals of the pulse output portion, to enable the at least two electrical connection terminals to be in electrical conduction with a human body through corresponding electrode groups to perform electric stimulation massage on the human body; and
wherein the controller is further configured to control the pulse output portion to switch conduction of the plurality of electrical connection terminals in a preset sequence, to sequentially conduct the plurality of electrode groups in a clockwise or counterclockwise direction to perform a circular massage on the human body.

2. The electric stimulation massager according to claim 1, wherein at least two electrode groups among the plurality of electrode groups are simultaneously conducted, wherein at least two electrode groups conducted at an earlier time and at least two electrode groups conducted later in sequence share at least one same electrode group.

3. The electric stimulation massager according to claim 1, wherein the controller is further configured to control the pulse output portion to output the electrical pulse through at least one electrical connection terminal among the plurality of electrical connection terminals and to control at least one electrical connection terminal among the plurality of electrical connection terminals to be grounded, to enable at least two electrical connection terminals to be in electrical conduction with one another.

4. The electric stimulation massager according to claim 3, wherein among the at least two electrical connection terminals that are in electrical conduction with one another, a number of electrodes connected to the at least one electrical connection terminal through which the electrical pulse is outputted is less than a number of electrodes connected to the at least one electrical connection terminal that is grounded.

5. The electric stimulation massager according to claim 4, wherein each of the plurality of electrode groups comprises the same number of electrodes, the controller is configured to control the pulse output portion to output the electrical pulse through one electrical connection terminal among the plurality of electrical connection terminals and to control two electrical connection terminals among the plurality of electrical connection terminals to be grounded, and each of the at least one electrode of each of the plurality of electrode groups has an area ranging from 1500 mm² to 2500 mm².

6. The electric stimulation massager according to claim 3, wherein among the at least two electrical connection terminals that are in electrical conduction with one another, a number of electrodes connected to the at least one electrical connection terminal through which the electrical pulse is outputted is equal to a number of electrodes connected to the at least one electrical connection terminal that is grounded.

7. The electric stimulation massager according to claim 3, wherein an operation process of the electric stimulation massager comprises an initial phase and an intermediate phase, wherein at least one electrode group connected to the at least one electrical connection terminal through which the electrical pulse is outputted serves as at least one output electrode group, and at least one electrode group connected to the at least one electrical connection terminal that is grounded serves as at least one input electrode group; in the initial phase, a distance between any one output electrode group among the at least one output electrode group and any one input electrode group among the at least one input electrode group that are simultaneously conducted is less than a preset distance; and in the intermediate phase, a distance between the at least one output electrode group and the at least one input electrode group that are simultaneously conducted is greater than the preset distance.

8. The electric stimulation massager according to claim 1, wherein the controller is configured to control the plurality of electrical connection terminals to be conducted sequentially at an interval ranging from 50 ms to 500 ms.

9. The electric stimulation massager according to claim 1, wherein the controller is configured to control the plurality of electrical connection terminals to be conducted sequentially at an interval ranging from 200 ms to 400 ms.

10. The electric stimulation massager according to claim 1, wherein an envelope waveform of the electrical pulse comprises a sine waveform, a triangular waveform, or a trapezoidal waveform.

11. The electric stimulation massager according to claim 1, wherein a frequency of the electrical pulse ranges from 1 kHz to 6 kHz, and a pulse width of the electrical pulse ranges from 40 μs to 250 μs.

12. The electric stimulation massager according to claim 1, wherein the electric stimulation massager is operable to be connected to a strap, each of the at least one electrode of each of the plurality of electrode groups is strip-shaped, and an included angle between a longitudinal direction of each of the at least one electrode of each of the plurality of electrode groups and a longitudinal direction of the strap ranges from 30 degrees to 60 degrees.

13. The electric stimulation massager according to claim 1, wherein the electrode is strip-shaped, and in each two adjacent electrode groups among the plurality of electrode groups, an included angle between a longitudinal direction of an electrode in one electrode group and a longitudinal direction of an electrode in another electrode group ranges from 60 degrees to 90 degrees.

14. An electric stimulation massaging device, comprising a strap and an electric stimulation massager connected to the strap, wherein the electric stimulation massager comprises:
a pulse output portion configured to output an electrical pulse, wherein the pulse output portion comprises a plurality of electrical connection terminals;
a base body;
a plurality of electrode groups disposed on one side of the base body, wherein each of the plurality of electrode groups comprises at least one electrode electrically connected to a same electrical connection terminal among the plurality of electrical connection terminals, and the plurality of electrode groups are spaced apart from one another and arranged in a loop; and
a controller configured to form conduction of at least two electrical connection terminals among the plurality of electrical connection terminals of the pulse output portion, to enable the at least two electrical connection terminals to be in electrical conduction with a human body through corresponding electrode groups to perform electric stimulation massage on the human body; and
wherein the controller is further configured to control the pulse output portion to switch conduction of the plurality of electrical connection terminals in a preset sequence, to sequentially conduct the plurality of electrode groups in a clockwise or counterclockwise direction to perform a circular massage on the human body.

15. The electric stimulation massaging device according to claim 14, wherein the electric stimulation massaging device is a waist massaging device, wherein the plurality of electrode groups are arranged in two loops, spaced apart from one another, and arranged to correspond to an erector spinae and a quadratus lumborum of the human body, to perform a circular massage on the erector spinae and the quadratus lumborum on both sides of the human body.

16. The electric stimulation massaging device according to claim 14, wherein at least two electrode groups among the plurality of electrode groups are simultaneously conducted, wherein at least two electrode groups conducted at an earlier time and at least two electrode groups conducted later in sequence share at least one same electrode group.

17. The electric stimulation massaging device according to claim 14, wherein the controller is further configured to control the pulse output portion to output the electrical pulse through at least one electrical connection terminal among the plurality of electrical connection terminals and to control at least one electrical connection terminal among the plurality of electrical connection terminals to be grounded, to enable at least two electrical connection terminals to be in electrical conduction with one another.

18. The electric stimulation massaging device according to claim 17, wherein among the at least two electrical connection terminals that are in electrical conduction with one another, a number of electrodes connected to the at least one electrical connection terminal through which the electrical pulse is outputted is less than a number of electrodes connected to the at least one electrical connection terminal that is grounded.

19. The electric stimulation massaging device according to claim 18, wherein each of the plurality of electrode groups comprises the same number of electrodes, the controller is configured to control the pulse output portion to output the electrical pulse through one electrical connection terminal among the plurality of electrical connection terminals and to control two electrical connection terminals among the plurality of electrical connection terminals to be grounded, and each of the at least one electrode of each of the plurality of electrode groups has an area ranging from 1500 mm² to 2500 mm².

20. The electric stimulation massaging device according to claim 17, wherein among the at least two electrical connection terminals that are in electrical conduction with one another, a number of electrodes connected to the at least one electrical connection terminal through which the electrical pulse is outputted is equal to a number of electrodes connected to the at least one electrical connection terminal that is grounded.
